(19) United States
(12) Patent Application Publication
(10) Pub. No.: US 2023/0009182 A1
(43) Pub. Date: Jan. 12, 2023

(54) SYSTEM FOR IDENTIFYING AND DIAGNOSING A DEGRADATION OF A PERFORMANCE METRIC ASSOCIATED WITH PROVIDING A SERVICE

(71) Applicant: Capital One Services, LLC, McLean, VA (US)

(72) Inventors: Mohamed SECK, Aubrey, TX (US); Eric SCHULTZ, Trappe, PA (US)

(21) Appl. No.: 17/305,423

(22) Filed: Jul. 7, 2021

(51) Int. Cl. G06Q 10/06; G06Q 30/00; G16H 10/60; G16H 40/20; G16H 50/70; G06F 17/18

(52) U.S. Cl. CPC G06Q 10/06393; G06Q 10/06375; G06Q 30/016; G16H 10/60; G16H 40/20; G16H 50/70; G06F 17/18; G06Q 10/1093

(57) ABSTRACT

In some implementations, a system may collect historical interaction information associated with previous service interactions involving users that received the service. The system may determine, based on the historical interaction information, an impact score associated with an attribute of a group of the users satisfies a degradation threshold. The system may receive a request involving a user receiving the service, wherein the request indicates that the user is associated with the attribute. The system may determine, based on the user being associated with the attribute and the impact score satisfying the degradation threshold, a parameter that reduces a probability that the user experiences the degradation of the performance metric when receiving the service. The system may perform an action associated with the parameter and the user.

### BACKGROUND

In many instances, a service provider is tasked with scheduling appointments to provide services for individuals. However, there are often many factors that can cause a duration of time required to provide a service to be different from the allotted time scheduled for a corresponding appointment. Furthermore, some factors may cause delays in a scheduled appointment starting on time, resulting in delays for other appointments on a schedule of the service provider or the individual.

### SUMMARY

Some implementations described herein relate to a system for identifying and diagnosing a degradation of a performance metric associated with a service. The system may include one or more memories and one or more processors communicatively coupled to the one or more memories. The one or more processors may be configured to maintain historical interaction information associated with previous service interactions involving users that received the service. The one or more processors may be configured to determine, based on the historical interaction information, an impact score associated with an attribute of a first group of the users interacting in a stage of the service. The one or more processors may be configured to analyze, based on the impact score satisfying a degradation threshold, a second group of the users to diagnose a cause of a member of the first group experiencing the degradation during one of the previous service interactions. The one or more processors may be configured to identify a difference associated with a parameter involving the first group receiving the service and the second group receiving the service. The one or more processors may be configured to perform, based on the difference satisfying a difference threshold, an action associated with the parameter and the first group of the users.

Some implementations described herein relate to a method for diagnosing a degradation of a performance metric of a service. The method may include collecting, by a device, historical interaction information associated with previous service interactions involving users that received the service. The method may include determining, by the device and based on the historical interaction information, an impact score associated with an attribute of a group of the users satisfies a degradation threshold. The method may include receiving, by the device, a request involving a user receiving the service, where the request indicates that the user is associated with the attribute. The method may include determining, by the device and based on the user being associated with the attribute and the impact score satisfying the degradation threshold, a parameter that reduces a probability that the user experiences the degradation of the performance metric when receiving the service. The method may include performing, by the device, an action associated with the parameter and the user.

Some implementations described herein relate to a non-transitory computer-readable medium that stores a set of instructions for a device. The set of instructions, when executed by one or more processors of the device, may cause the device to receive a request associated with a user receiving a service. The set of instructions, when executed by one or more processors of the device, may cause the device to determine, based on previous service interactions, an impact score associated with the attribute and a performance metric associated with providing the service. The set of instructions, when executed by one or more processors of the device, may cause the device to select, based on the impact score satisfying a degradation threshold and the historical service information, a parameter associated with providing the service. The set of instructions, when executed by one or more processors of the device, may cause the device to cause a provider system to be configured in association with the parameter to provide the service for the user.

### BRIEF DESCRIPTION OF THE DRAWINGS

FIGS. 1A-1B are diagrams of an example implementation relating to a system, as described herein, for identifying and diagnosing a degradation of a performance metric associated with providing a service.

FIGS. 2A-2C are diagrams of an example implementation related to identifying and diagnosing a degradation of a performance metric associated with providing a service, as described herein.

FIG. 3 is a diagram of an example environment in which systems and/or methods described herein may be implemented.

FIG. 4 is a diagram of example components of one or more devices of FIG. 3.

FIG. 5 is a flowchart of an example process relating to identifying and diagnosing a degradation of a performance metric associated with providing a service.

### DETAILED DESCRIPTION

The following detailed description of example implementations refers to the accompanying drawings. The same reference numbers in different drawings may identify the same or similar elements.

A scheduling system typically involves allocating a time slot of a calendar for multiple individuals to receive services from a service provider (e.g., a healthcare organization, a financial institution, food service organization, a vehicle services center, amongst other examples). For example, a service provider may maintain and/or manage the scheduling system by indicating availability to provide a service, and a user may use the system to reserve a time slot to receive the service from the service provider. Timing for providing a service may be somewhat unpredictable. For example, various factors or events may prevent the service provider from staying on schedule. Such unpredictability causes a degraded user experience, degraded production, and/or degraded efficiency. Some systems or service providers may attempt to track timing for providing a service in order to retroactively make adjustments to more accurately predict timing for providing the service, however, this may result in further degraded performance of the service provider (e.g., because changes to past performance cannot be made and the changes may not solve the problems involved in providing the service). Moreover, services that involve multiple stages (e.g., separate periods of time for receiving the service or multiple sub-services) add further complexity with respect to issues involved in providing a service and/or predicting timing associated with providing the service.

Some implementations described herein provide a service management system that is configured to receive or collect historical interaction information associated with a service provider providing a service to various groups of users. The historical interaction information may include information associated with timing of users receiving a service along with corresponding attributes of the users and parameters involved in receiving the service. The attribute may include individual characteristics of the users, such as age, location, gender, health condition, among other physical or health-related characteristics. The parameters involved in receiving the service may involve or be associated with using certain systems, devices, processes, technologies, or representatives that provided the service to the users at individual stages of providing the service.

The service management system may analyze historical interactions involving the groups of users that received the service via the historical interactions and determine that an attribute of a group receiving the service is associated with a degradation of service (e.g., based on an impact score associated with timing of the group receiving the service). The service management system may identify a parameter of another group that received the service without experiencing a degradation of service and perform an action based on the parameter and the group.

In this way, the service management system may proactively identify a degradation of service involving a particular group and diagnose or identify a factor that improves performance of providing the service for individuals associated with the group (e.g., individuals that share the attribute). Accordingly, the service management system may receive, maintain, and analyze various factors involved in receiving a service, including multiple stages of receiving the service, to maintain and/or improve performance with respect to providing a service, thereby leading to improved predictability for providing the service. Therefore, the system, as described herein may reduce downtime or other inefficiencies, improve a user experience, reduce waste with respect to scheduling (or allocating) resources for providing a service whether the resources are not necessary or needed.

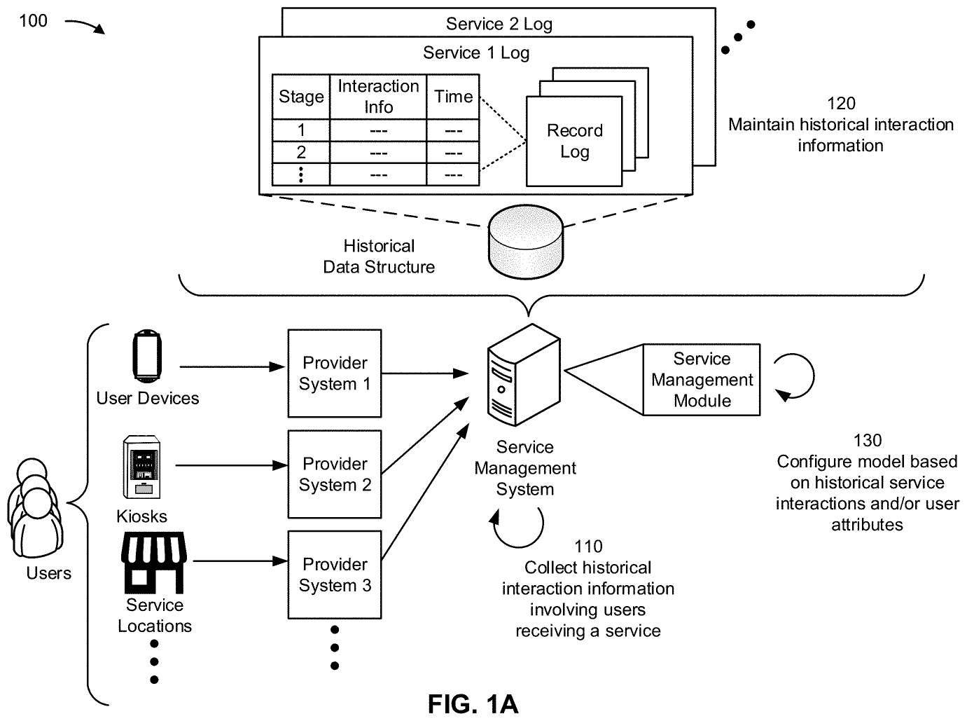

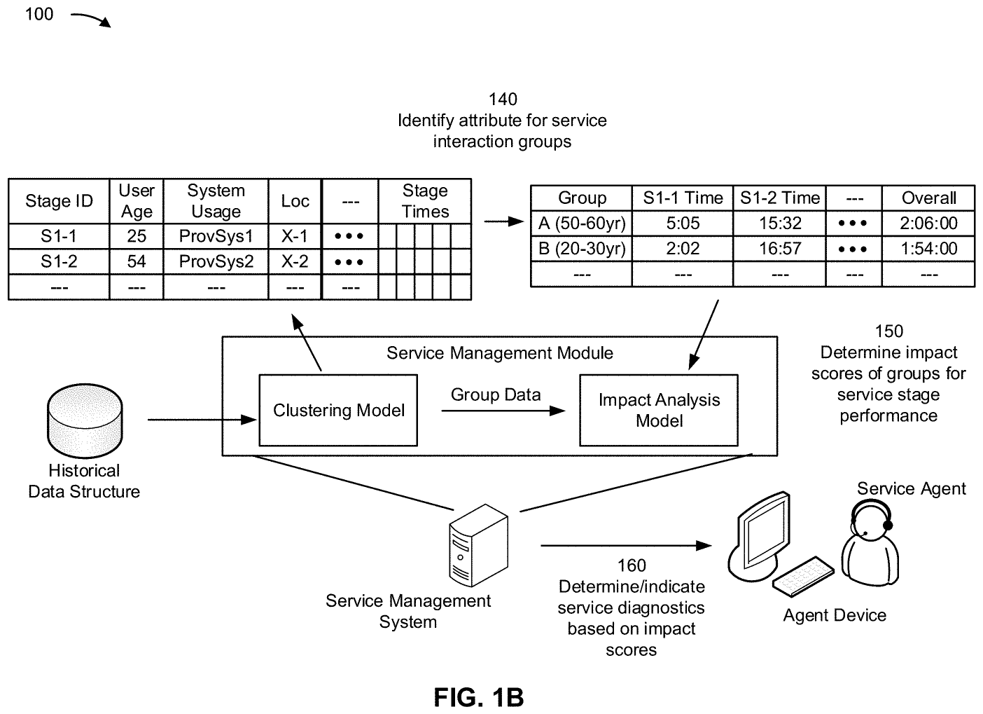

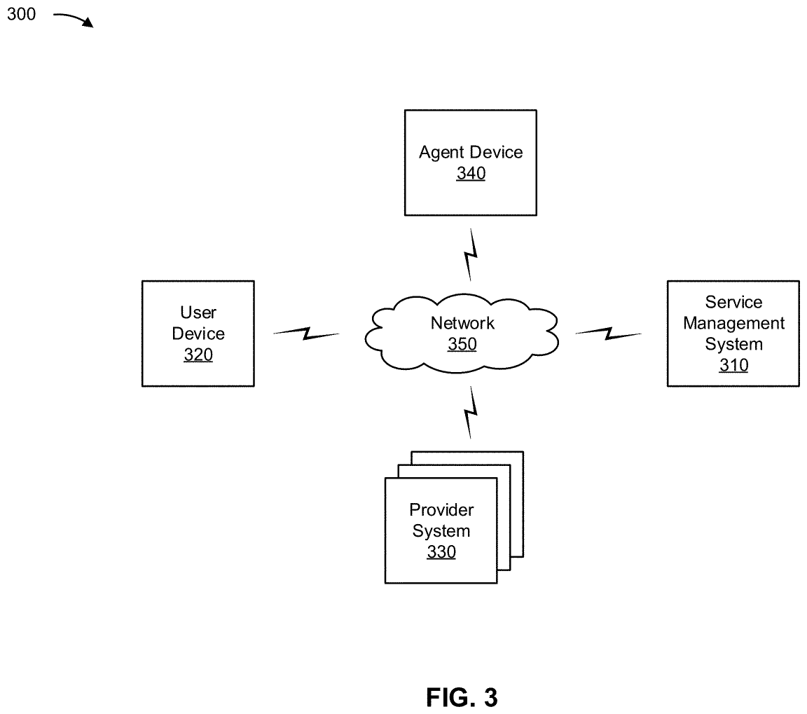

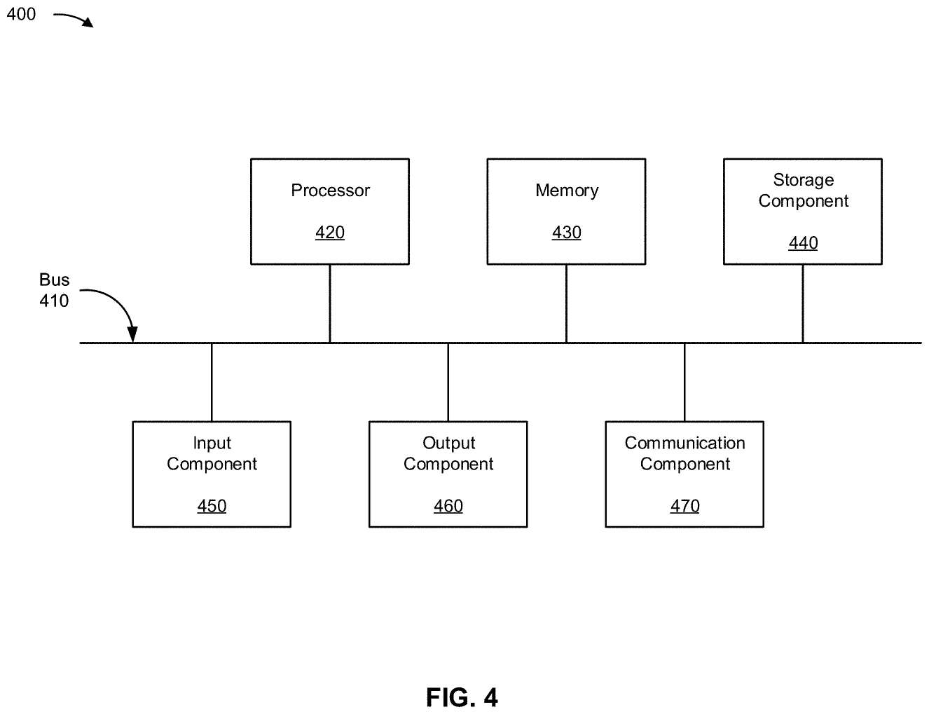

FIGS. 1A-1B are diagrams of an example implementation 100 associated with a system for identifying and diagnosing a degradation of a performance metric associated with providing a service. As shown in FIGS. 1A-1B, example implementation 100 includes a service management system, provider systems (e.g., that are associated with user devices, kiosks, and/or service locations), and an agent device associated with a service agent. These devices are described in more detail below in connection with FIG. 3 and FIG. 4.

As described herein, the service management system, the one or more provider systems, and/or an application installed on the user device that facilitate a service interaction may be associated with a service provider (e.g., an individual and/or organization). The service provider may provide one or more services for the users (e.g., healthcare services, computing services, telecommunication services, network security services, financial services, product services, maintenance services, warranty services, retail services, transportation services, and/or other types of services).

As shown in FIG. 1A, and by reference number 110, the service management system collects historical interaction information involving the users receiving a service. For example, the service management system may receive historical data that is associated with previous service interactions between users and a service provider. The historical interaction information may identify dates and/or times of previous service interactions, types of services that were involved in the previous service interactions, and/or timing information associated with durations of stages of providing services during the previous service interactions.

The service management system may receive and/or obtain the historical interaction information based on monitoring and/or collecting information from the users and/or from the service provider in association with services being performed (e.g., via a related survey and/or via a related notification). Additionally, or alternatively, the service management system may monitor one or more of the provider systems, identify ongoing service interactions via the individual provider systems, obtain identifiers of user accounts involved in the service interactions, and record and/or store information associated with the previous service interactions within a historical data structure, as shown. More specifically, the service management system may store the historical interaction information in service logs associated with various services provided by the service provider.

As further shown in FIG. 1A, and by reference number 120, the service management system maintains the historical interaction information. In some implementations, the service management system may maintain the historical interaction information according to information received about the users and/or based on user account information associated with the users. For example, the service management system may maintain the historical interaction information based on receiving information that indicates that a user has engaged or is engaging in an ongoing service interaction (e.g., based on an application of the user device and/or the provider systems indicating that the user is receiving a service or is within one or more stages for receiving the service).

As shown, the historical interaction information may be maintained within a service log that includes a plurality of record logs associated with individual users receiving the service. A record log of receiving a service (e.g., during a previous service interaction) that may include information associated with various stages of receiving the service. Interaction information (Interaction Info) and time information (Time) may be mapped to the stages of the service. The interaction information may include information associated with the user receiving a service or engaging in a service interaction. For example, the interaction information may identify user information (e.g., information that identifies one or more attributes of the user, an account of the user, a location of the user, a device associated with the user, among other examples) and/or parameters involved in receiving the service (e.g., a location involved in the stage of the service, a resource utilized during the service interaction, a provider system that provided information associated with the stage of the service, among other examples). The timing information may indicate a start time, end time, and/or duration of the stage of the service during the previous service interaction.

As a more specific example, for a service involving a healthcare facility, a first stage of receiving the service may involve checking into an office to receive the service, a second stage may involve being led to a room of the healthcare facility to receive the service, and a third stage may involve receiving treatment from a healthcare professional, and so on. Accordingly, the interaction information for the first stage may indicate which of the one or more provider systems were used to check into a healthcare facility. In such a case, the interaction information for the second stage may include information associated with staffing at the healthcare facility and/or whether the healthcare facility was adequately staffed. Furthermore, the interaction information for the third stage may indicate which types of treatment were provider and/or which devices were used to provide the treatment, among other examples.

The provider systems may be configured to report and/or provide, to the service management system, information associated with receiving the service and/or providing the service. For a service that involves multiple stages, a provider system may identify which stage of the service has been performed or is being performed (e.g., based on user inputs from the users and/or service providers) during a service interaction. Accordingly, via the provider systems, the service management system may obtain historical interaction information that is associated with previous service interactions involving groups of users, as described elsewhere herein.

As further shown in FIG. 1A, and by reference number 130, the service management system configures a model based on the historical service interactions and/or user attributes. For example, the service management system may configure one or more models of the service management module, such as a clustering model and/or an impact analysis model described elsewhere herein. More specifically, the service management system may configure settings for the clustering model and/or the impact analysis model.

In some implementations, the service management system may configure the models according to certain attributes associated with the users involved in the previous service interactions. For example, the service management system may receive information that designates various types of user attributes for analysis. Additionally, or alternatively, the service management system may configure the service management module to identify values for various attributes of the users from the historical data structure and/or account information associated with user accounts of the users. The designated attributes may include or be associated with one or more age ranges of users, one or more health statuses (or conditions) of users, one or more locations associated with users, one or more races of users, one or sexes or genders of users, among other examples. Additionally, or alternatively, the service management system may configure the models to identify various performance metrics associated with receiving a service and/or associated with certain stages of receiving a service. For example, a performance metric may include a duration of time for receiving or providing a service (or a certain stage of a service), an accuracy with respect to predicting a duration of time for receiving or providing the service, a measure of success with respect to providing a service, a level of user satisfaction associated with receiving the service, among other examples.

The service management system may configure the service management module to utilize the attributes and/or performance metrics for an impact analysis associated with the attributes. For example, the service management system may receive the attributes within configuration information (e.g., information within one or more administrator inputs) that identifies the attributes and/or performance metrics. The configuration information may designate which attributes are to be mapped to which performance metrics for the impact analysis. Additionally, or alternatively, the service management system may configure the service management module to identify different parameters for receiving or providing the service, which may be indicated in the configuration information. Accordingly, the event scheduling system may designate one or more event statuses that are to be mapped to values of the performance parameter for the linear regression analysis.

In this way, the service management system may configure one or more models, as described herein, to analyze the historical interaction information associated with receiving or providing a service to a user. Furthermore, the service management system may configure the one or more models to be dynamically updated (e.g., based on receiving information associated with subsequent service interactions or ongoing service interactions involving the provider systems), as described elsewhere herein.

As shown in FIG. 1B, and by reference number 140, the service management system identifies attributes for service interaction groups. As shown in FIG. 1B, the service management module may include a clustering model and an impact analysis model. The clustering model may sort service records according to one or more sets of attributes of the users for an impact analysis. For example, the clustering model may be configured to identify one or more attributes of a group that receives a degradation in a performance metric relative to other groups. As a more specific example, the clustering model may be configured to identify an age group of users that experiences (or has experienced during previous service interactions) relatively longer service times at one or more stages of a service than other groups. In example implementation 100, Group A may experience longer times for Stage S1-1 and shorter times for S1-2 than Group B. The clustering model may determine the times for Stage S1-1 and S1-2 based on respective record logs involving the users with ages within a range of values associated with Group A (50-60 years) and Group B (20-30 years).

Accordingly, the service management system, via the clustering model, may identify a first group (Group A) as being associated with a first attribute (e.g., a first age range of 50-60 years old) and a second group (Group B) as being associated with a second attribute (e.g., a second age range of 20-30 years old). Moreover, as indicated in this example, the range of values of a first attribute may be mutually exclusive from a range of values of a second attribute. However, in some implementations, values for a particular type of attribute may not be mutually exclusive from one another (e.g., a user may be associated with multiple locations, multiple races, among other examples). While certain examples are described herein in connection with age as an attribute, other examples are possible, including groups of users that share multiple types of attributes with one another.

As further shown in FIG. 1B, and by reference number 150, the service management system determines impact scores of groups for service stage performance. The service management system may determine the impact score via the impact analysis model. In some implementations, the impact analysis model includes or is associated with a linear regression model. Accordingly, the impact score may be determined using a linear regression analysis of attributes of the users. For example, the impact analysis model may be configured to analyze attributes of the users that are mapped to certain performance metrics and/or values of performance metrics. The impact analysis model may be configured to analyze the groups based on values that indicate a degradation of the performance metric for a particular group in order to determine whether the attribute is indicative of a cause of the degradation of the performance metric. Accordingly, the impact score may be indicative of whether the attribute is associated with corresponding interactions of the previous service interaction involving the degradation of the performance metric. Additionally, or alternatively, the impact score may be associated with a parameter involved in service interactions associated with a group of users.

In some implementations, the performance metric is associated with a particular stage of the multiple stages. For example, as shown in FIG. 1B, the performance metric may correspond to a length (or duration) of a period of time associated with a particular stage associated with one or more of the previous service interactions. The value of the performance metric may include an average or other value determined from the linear regression analysis. In this way, the service management system (and/or impact analysis model) may identify performance metrics associated with one or more groups of the users involved in the previous service interactions to determine an impact score associated with certain attributes of the groups.

In some implementations, to determine the impact score, the service management system, via the impact analysis model, may identify a difference associated with a parameter involving a first group (e.g., Group A) receiving a service and a second group (e.g., Group B) receiving the service. In some implementations, the analysis may be based on a particular stage of the service. For example, referring to FIG. 1B, for Stage S1-1, an impact score may be determined for an attribute of Group A based on one or more parameters involved with receiving the service. As shown in FIG. 1B, a user (age 25) from Group A may have used a first provider system (ProvSys1 corresponding to Provider System 1 of FIG. 1A) and a user (age 54) from Group B may have used a second provider system (ProvSys2 corresponding to Provider System 2 of FIG. 1A). Accordingly, the impact analysis model (e.g., via a linear regression analysis) may identify an impact score for using the first provider system based on other users (e.g., of Group A, Group B, or any other group) that used the first provider system for Stage S1-1. Additionally, or alternatively, the impact analysis model may determine the impact score for using the second provider system based on other users that use the second provider system for Stage S1-1.

The impact analysis model may determine an impact that using the first provider system or the second provider system may have on certain groups (that have certain attributes) receiving a degradation in performance based on a difference associated with usage of provider systems during Stage S1-1. For example, if a majority of Group A utilizes the first provider system to receive the service and a majority of Group B utilizes the second provider system, the impact score may indicate that Group B may be experiencing the degradation in performance due to utilizing the second provider system rather than the first provider system (e.g., the older age group may prefer checking-in at a kiosk rather than using a user device). Additionally, or alternatively, the difference associated with the parameter involving the usage of the provider systems is indicative of a higher percentage of Group A utilizing the first provider system for receiving the service than Group B.

As further shown in FIG. 1B, and by reference number 160, the service management system determines and/or indicates service diagnostics based on the impact scores. For example, based on an impact score satisfying a degradation threshold, the service management system may analyze parameters associated with the previous service interactions to diagnose a cause of a member of a group experiencing a degradation of service during one of the previous service interactions. The degradation threshold may correspond to comparison of a performance metric associated with a first group and a second group. For example, a degradation threshold for a degradation of performance based on a length of a stage of a service may be based on an average length of all users (or users of a particular group) receiving the service (or being involved in a particular stage of receiving the service) during the previous service interactions.

In some implementations, if a difference associated with a parameter (e.g., a difference in usage of the provider systems in example implementation 100) satisfies a difference threshold, then the service management system may determine that the parameter may be a cause for a degradation of a performance metric (e.g., a length of the stage of receiving the service). The value of the difference threshold that is to be satisfied may be based on a type of the parameter. For example, a difference threshold for usage of one type of provider system may be different from a difference threshold for usage of another type of provider system. Additionally, or alternatively, a difference threshold for comparing provider systems may be different from a difference threshold for comparing another type of parameter, such as location where the service was received (or a location associated with a stage of the service), a representative or type of representative that was involved in the stage of the service, and/or the like.

Additionally, or alternatively, the difference threshold may be based on a performance metric that was analyzed in association with determining the impact score. For example, if a difference between a performance metric associated with a first group and a second group is relatively high, then a difference threshold associated with a parameter may be configured to also be relatively high. On the other hand, if a difference between a performance metric associated with a first group and a second group is relatively low, then a difference threshold associated with a parameter may be configured to also be relatively low. In this way, the service management system, via the service management module, may determine diagnostic information associated with a degradation of a performance metric involving a particular group of users (e.g., a group of users that is associated with a particular attribute).

The service management system may perform one or more actions associated with the diagnostic information and/or the group of users. For example, the service management system may perform an action to proactively improve the performance metric for a member associated with the group that permits a service request, as described elsewhere herein. In connection with example implementation 100, the service management system may be configured to instruct the agent (via the agent device) to recommend or suggest that another member of Group B should use the first provider system (rather than the second provider system), in order to prevent a degradation of performance during Stage S1-1 of receiving the service. Additionally, or alternatively, the service management system may be configured to instruct the other member to use the first provider system.

In this way, the service management system may provide, to an agent device, a notification that indicates that members associated with a particular attribute are to receive the service in association with a system that is associated with the parameter (and/or a process that is associated with the parameter).

As indicated above, FIGS. 1A-1B are provided as an example. Other examples may differ from what is described with regard to FIGS. 1A-1B. The number and arrangement of devices shown in FIGS. 1A-1B are provided as an example. In practice, there may be additional devices, fewer devices, different devices, or differently arranged devices than those shown in FIGS. 1A-1B. Furthermore, two or more devices shown in FIGS. 1A-1B may be implemented within a single device, or a single device shown in FIGS. 1A-1B may be implemented as multiple, distributed devices. Additionally, or alternatively, a set of devices (e.g., one or more devices) shown in FIGS. 1A-1B may perform one or more functions described as being performed by another set of devices shown in FIGS. 1A-1B.

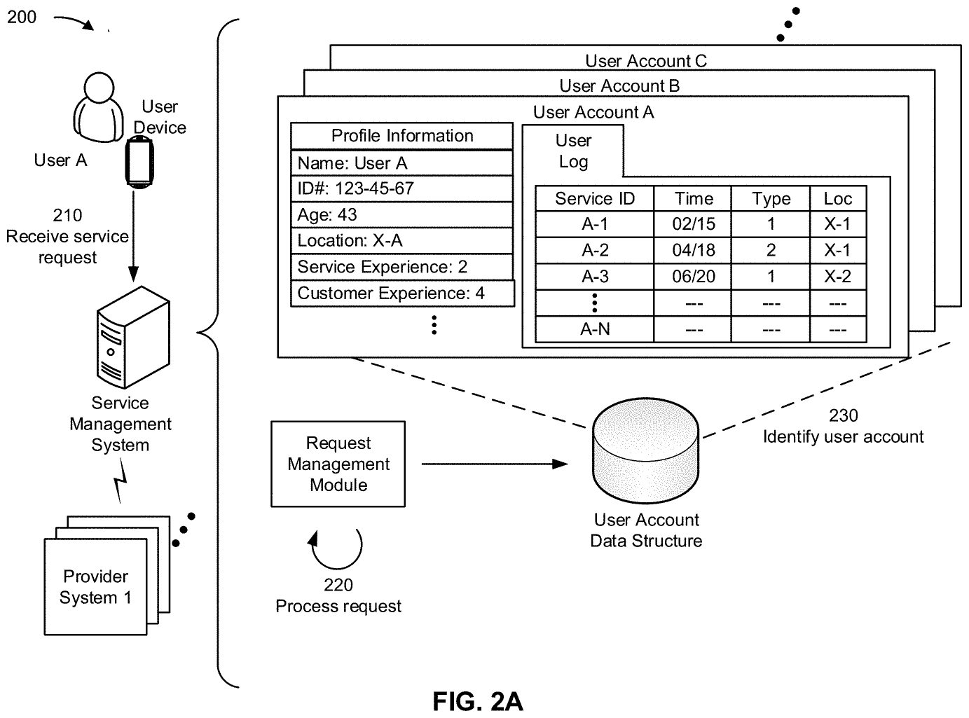

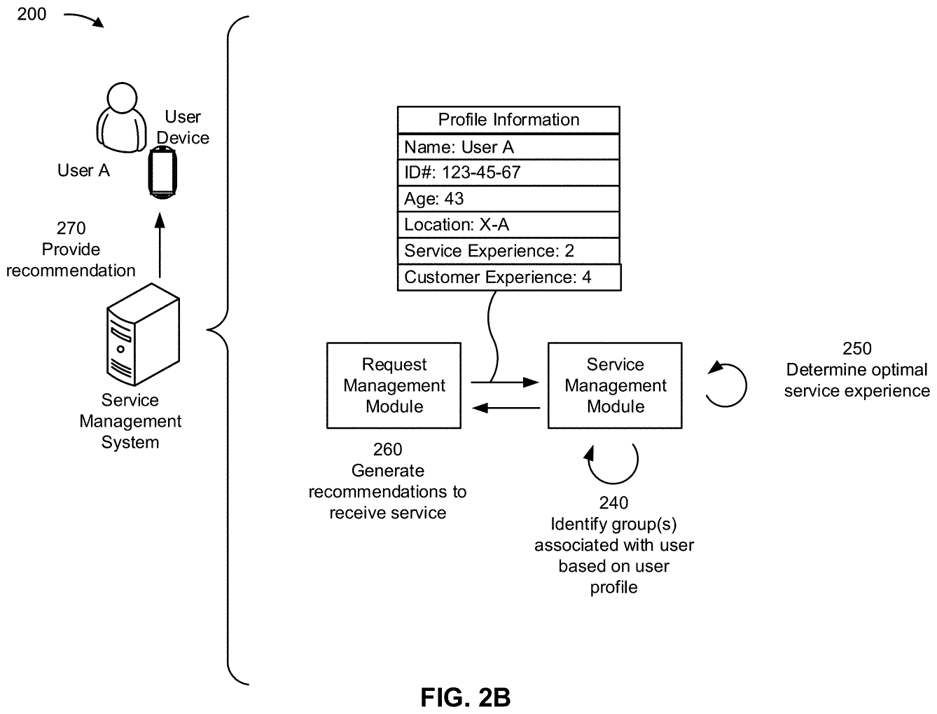

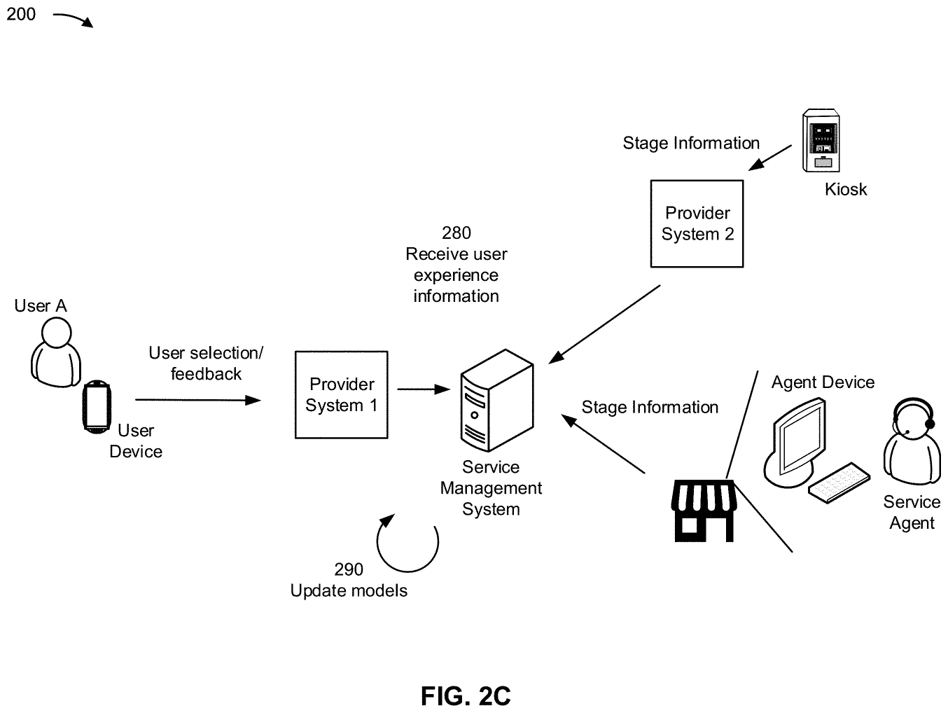

FIGS. 2A-2C are diagrams of an example implementation 200 associated with identifying and diagnosing a degradation of a performance metric associated with providing a service. As shown in FIGS. 2A-2C, example implementation 200 includes a service management system, one or more provider systems, a user device associated with a user (User A). These devices may be associated with corresponding devices described in connection with example implementation 100.

As shown in FIG. 2A, and by reference number 210, the service management system receives a service request from the user device. For example, the service request may correspond to a request that the service provider provide a service to user (User A). The request may include any suitable message and/or notification (e.g., a text message, a chat message of an instant message interface, an audio message, a video message, a voice call, and/or an electronic mail message, among other examples). The request may be received prior to the service management system performing action associated with an attribute of the user. For example, such an action may be performed to provide a notification that indicates that the user is to receive the service using a particular system or process associated with the parameter, as described elsewhere herein.

The request may include information that identifies User A (e.g., a name of User A and/or an account identifier of User Account A). Accordingly, the request may indicate one or more attributes of the user. Additionally, or alternatively, the request may include service information that identifies a service and/or a service type that is to be provided to the user, by the service provider, during or in association with the service interaction. For example, referring to the healthcare facility example described above, the service may include receiving a healthcare service. In such a case, the service may involve multiple stages, as described above.

As further shown in FIG. 2A, and by reference number 220, the service management system processes the request. For example, the service management system may process the request via a request management module that serves as an interface or request receiving module of the service management system. The request management module may utilize any suitable technique to process the request to identify and/or interpret the service information, user information, and/or other types of information associated with the request. For example, the service management system may utilize one or more of a natural language processing technique, an optical character recognition technique, a speech-to-text technique, a parsing technique, a sentiment analysis, and/or the like. In some implementations, the request management module may include and/or be associated with a chatbot, a voice call system, a call service system, a touchtone service system, and/or the like. The service management system may process the service request to identify the user, to identify a type of service that is to be provided to the user, and/or to identify a type of service interaction that is being requested by the user.

As further shown in FIG. 2A, and by reference number 230, the service management system may identify a user account associated with the user. For example, based on processing the request and/or identifying an identifier of User A, the request management module may identify information and/or data associated with User Account A in the user account data structure. Correspondingly, as shown, the request management module may access profile information of User A (including one or more attributes associated with User A). In some implementations, the service management system may determine and/or update one or more of the attributes based on receiving the service request (e.g., if an attribute is newly identified).

As shown in FIG. 2B, and by reference number 240, the service management system may identify one or more groups associated with the user based on the user profile. For example, the service management system, via the request management module, may analyze the user account to identify one or more attributes of the user. Additionally, or alternatively, the request management module may identify the attribute of the user based on information received in the request. For example, the request management module may process the request to determine whether the user is associated with a particular group that is associated with a degradation of performance of the service (or a stage of the performance). For example, the service management system may determine that the user is associated with a first group of users that received a degradation in performance during previous service interactions (e.g., relative to another group of users, such as a second group of users). The first group may be based on an age range of an age range of members of the first group, a health status of members of the first group, a location associated with members of the first group, and/or other user attributes.

As further shown in FIG. 2B, and by reference number 250, the service management system may determine an optimal service experience. For example, the service management system, via the service management module, may determine an optimal service experience according to the impact analysis model described above and one or more of the attributes of the first group. More specifically, the service management system may determine, based on previous service interactions involving users of the first group, an impact score associated with the attribute and a performance metric associated with providing the service. As described above, the impact score may be determined based on historical service information that indicates that the first group of the users were associated with the attribute when receiving the service.

The service management system may determine the optimal service experience based on based on the user being associated with the attribute and an impact score satisfying a degradation threshold. For example, the service management system may determine a parameter for receiving the service (e.g., using a particular system and/or process) according to the optimal service experience.

The service management system may determine the parameter using the impact analysis model based on an analysis of the first group and a second group (e.g., a comparison of performance metrics and/or parameters used in receiving the services), as described elsewhere herein. The second group of the users did not experience a degradation of the performance metric. In some implementations, the parameter may be identified based on the second group of users not experiencing the degradation of the performance. Additionally, or alternatively, the service management system may select the parameter for the user to receive an optimal service experience. For example, as described herein, the service management system may select the parameter based on an impact score associated with parameter satisfying a degradation threshold (and/or based on the historical service information indicating that the second group received the service according to the parameter). In this way, the parameter for the optimal service experience may be identified based on the historical service information indicating that a second group of the users received the service in accordance with the parameter without experiencing the degradation of the performance metric.

In this way, the service management system may determine a parameter for optimizing a service experience for a user to permit the service management system to perform an action associated with the parameter and the user.

As further shown in FIG. 2B, and by reference number 260, the service management system generates a recommendation to receive the service. For example, the service management system may generate a notification that instructs the user to utilize a system and/or process that is associated with the parameter. More specifically, the service management system may generate the notification to indicate that the user should use a particular system that is typically not used by other users of the first group (or other users with a shared attribute of the user).

As further shown in FIG. 2B, and by reference number 270, the service management system provides the recommendation to the user. For example, the service management system may provide the recommendation to a user device that sent the service request and/or a user device that is identified in the user profile. In some implementations, the service management system may cause an indicated provider system to be configured in association with the parameter to provide the service for the user. Additionally, or alternatively the service management system may provide, to the agent device, a notification that indicates that the user is to receive the service in association with the parameter (and/or the provider system).

In some implementations, the service management system may send a feedback request associated with a selected parameter (e.g., associated with a selected provider system) for receiving the service. For example, the feedback request may solicit the user to indicate whether a requested, suggested, and/or assigned provider system was available to the user and/or useful in facilitating a satisfactory level of service for a particular stage of receiving the service. In this way, the service management system may request, via a user device associated with a member of the first group (User A), feedback associated with the member receiving the service during a corresponding service interaction.

As further shown in FIG. 2B, and by reference number 280, the service management system may receive user experience information. For example, as shown, the service management system may receive a user selection from the user (e.g., a selection of a particular provider system for receiving the service) and/or feedback associated with the user receiving the service. The service management system may receive the user selection and/or the feedback from a first provider system and/or a user device associated with the user.

Additionally, or alternatively, the service management system may receive stage information from a second provider system and/or a kiosk. The stage information may be associated with a particular stage of the user receiving the service. For example, the stage information may indicate timing associated with the user receiving the service during the stage. In some implementations, the agent device and/or a provider system associated with a service location, may be used to provide stage information associated with the service experience. This way, the service management system may continuously receive information associated with service interactions involving a service and/or users of various groups to permit the service management system to update one or more models for identifying and/or diagnosing a degradation in performance in connection with the service and/or attributes of the groups.

As further shown in FIG. 2B, and by reference number 290, the service management system may update the models. For example, the service management system may update the models by reconfiguring the clustering model to include newly received attributes of the users and/or service interaction information associated with users receiving a service. Furthermore, the impact analysis model may be updated and/or configured according to provider systems available to receive a service and/or a response associated with feedback from the user. For example, the impact analysis model may be updated to adjust values for performance metrics associated with certain groups and/or parameters that members of the groups used to receive the service and/or that the service provider used to provide the service.

Accordingly, as described herein, a service management system is provided to robustly identify and/or diagnose a degradation in a performance metric associated with providing or receiving a service and/or associated with a stage of providing a service (and/or a stage of receiving the service). In this way, a service provider may proactively improve one or more systems for providing the service and/or manners in which groups of users are to receive certain services, thereby enhancing predictability with respect to providing the service and avoiding a waste of resources caused by other systems that are not configured as described herein.

As indicated above, FIGS. 2A-2C are provided as an example. Other examples may differ from what is described with regard to FIGS. 2A-2C. The number and arrangement of devices shown in FIGS. 2A-2C are provided as an example. In practice, there may be additional devices, fewer devices, different devices, or differently arranged devices than those shown in FIGS. 2A-2C. Furthermore, two or more devices shown in FIGS. 2A-2C may be implemented within a single device, or a single device shown in FIGS. 2A-2C may be implemented as multiple, distributed devices. Additionally, or alternatively, a set of devices (e.g., one or more devices) shown in FIGS. 2A-2C may perform one or more functions described as being performed by another set of devices shown in FIGS. 2A-2C.

FIG. 3 is a diagram of an example environment 300 in which systems and/or methods described herein may be implemented. As shown in FIG. 3, environment 300 may include a service management system 310, a user device 320, one or more provider systems 330, an agent device 340, and a network 350. Devices of environment 300 may interconnect via wired connections, wireless connections, or a combination of wired and wireless connections.

The service management system 310 includes one or more devices capable of receiving, generating, storing, processing, providing, and/or routing information associated with identifying and diagnosing a degradation of a performance metric associated with providing a service, as described elsewhere herein. The service management system 310 may include a communication device and/or a computing device. For example, the service management system 310 may include a server, such as an application server, a client server, a web server, a database server, a host server, a proxy server, a virtual server (e.g., executing on computing hardware), or a server in a cloud computing system. In some implementations, the service management system 310 includes computing hardware used in a cloud computing environment.

The user device 320 includes one or more devices capable of receiving, generating, storing, processing, and/or providing information associated with a service request (and/or service interaction request), receiving a service, and/or tracking timing associated with stages of receiving the service, as described elsewhere herein. The user device 320 may include a communication device and/or a computing device. For example, the user device 320 may include a wireless communication device, a mobile phone, a user equipment, a laptop computer, a tablet computer, a desktop computer, a wearable communication device (e.g., a smart wristwatch, a pair of smart eyeglasses, a head mounted display, or a virtual reality headset), or a similar type of device.

The provider system 330 includes one or more devices capable of receiving, generating, storing, processing, providing, and/or routing information associated with facilitating and/or managing a service interaction, as described elsewhere herein. The provider system 330 may include a communication device and/or a computing device. For example, the provider system 330 may include a server, such as an application server, a client server, a web server, a database server, a host server, a proxy server, a virtual server (e.g., executing on computing hardware), or a server in a cloud computing system. In some implementations, the provider system 330 includes computing hardware used in a cloud computing environment.

The agent device 340 includes one or more devices capable of receiving, generating, storing, processing, and/or providing information associated with an agent-side version of an application that is used for managing a service and/or tracking timing associated with stages of receiving the service, as described elsewhere herein. The agent device 340 may include a communication device and/or a computing device. For example, the agent device 340 may include a wireless communication device, a mobile phone, a user equipment, a laptop computer, a tablet computer, a desktop computer, a wearable communication device (e.g., a smart wristwatch, a pair of smart eyeglasses, a head mounted display, or a virtual reality headset), or a similar type of device.

The network 350 includes one or more wired and/or wireless networks. For example, the network 350 may include a wireless wide area network (e.g., a cellular network or a public land mobile network), a local area network (e.g., a wired local area network or a wireless local area network (WLAN), such as a Wi-Fi network), a personal area network (e.g., a Bluetooth network), a near-field communication network, a telephone network, a private network, the Internet, and/or a combination of these or other types of networks. The network 350 enables communication among the devices of environment 300.

The number and arrangement of devices and networks shown in FIG. 3 are provided as an example. In practice, there may be additional devices and/or networks, fewer devices and/or networks, different devices and/or networks, or differently arranged devices and/or networks than those shown in FIG. 3. Furthermore, two or more devices shown in FIG. 3 may be implemented within a single device, or a single device shown in FIG. 3 may be implemented as multiple, distributed devices. Additionally, or alternatively, a set of devices (e.g., one or more devices) of environment 300 may perform one or more functions described as being performed by another set of devices of environment 300.

FIG. 4 is a diagram of example components of a device 400, which may correspond to the service management system 310, the user device 320, and/or the agent device 340. In some implementations, the service management system 310, the user device 320, and/or the agent device 340 may include one or more devices 400 and/or one or more components of device 400. As shown in FIG. 4, device 400 may include a bus 410, a processor 420, a memory 430, a storage component 440, an input component 450, an output component 460, and a communication component 470.

Bus 410 includes a component that enables wired and/or wireless communication among the components of device 400. Processor 420 includes a central processing unit, a graphics processing unit, a microprocessor, a controller, a microcontroller, a digital signal processor, a field-programmable gate array, an application-specific integrated circuit, and/or another type of processing component. Processor 420 is implemented in hardware, firmware, or a combination of hardware and software. In some implementations, processor 420 includes one or more processors capable of being programmed to perform a function. Memory 430 includes a random access memory, a read only memory, and/or another type of memory (e.g., a flash memory, a magnetic memory, and/or an optical memory).

Storage component 440 stores information and/or software related to the operation of device 400. For example, storage component 440 may include a hard disk drive, a magnetic disk drive, an optical disk drive, a solid state disk drive, a compact disc, a digital versatile disc, and/or another type of non-transitory computer-readable medium. Input component 450 enables device 400 to receive input, such as user input and/or sensed inputs. For example, input component 450 may include a touch screen, a keyboard, a keypad, a mouse, a button, a microphone, a switch, a sensor, a global positioning system component, an accelerometer, a gyroscope, and/or an actuator. Output component 460 enables device 400 to provide output, such as via a display, a speaker, and/or one or more light-emitting diodes. Communication component 470 enables device 400 to communicate with other devices, such as via a wired connection and/or a wireless connection. For example, communication component 470 may include a receiver, a transmitter, a transceiver, a modem, a network interface card, and/or an antenna.

Device 400 may perform one or more processes described herein. For example, a non-transitory computer-readable medium (e.g., memory 430 and/or storage component 440) may store a set of instructions (e.g., one or more instructions, code, software code, and/or program code) for execution by processor 420. Processor 420 may execute the set of instructions to perform one or more processes described herein. In some implementations, execution of the set of instructions, by one or more processors 420, causes the one or more processors 420 and/or the device 400 to perform one or more processes described herein. In some implementations, hardwired circuitry may be used instead of or in combination with the instructions to perform one or more processes described herein. Thus, implementations described herein are not limited to any specific combination of hardware circuitry and software.

The number and arrangement of components shown in FIG. 4 are provided as an example. Device 400 may include additional components, fewer components, different components, or differently arranged components than those shown in FIG. 4. Additionally, or alternatively, a set of components (e.g., one or more components) of device 400 may perform one or more functions described as being performed by another set of components of device 400.

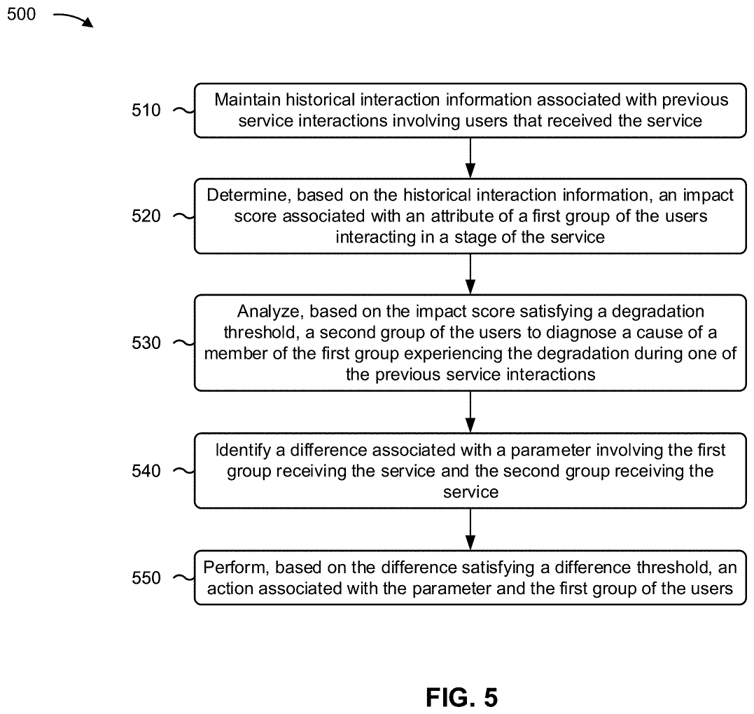

FIG. 5 is a flowchart of an example process 500 associated with identifying and diagnosing a degradation of a performance metric associated with providing a service. In some implementations, one or more process blocks of FIG. 5 may be performed by a service management system (e.g., the service management system 310). In some implementations, one or more process blocks of FIG. 5 may be performed by another device or a group of devices separate from or including the service management system, such as the user device 320, the provider system 330, and/or the agent device 340. Additionally, or alternatively, one or more process blocks of FIG. 5 may be performed by one or more components of device 400, such as processor 420, memory 430, storage component 440, input component 450, output component 460, and/or communication component 470.

As shown in FIG. 5, process 500 may include maintaining historical interaction information associated with previous service interactions involving users that received the service (block 510). As further shown in FIG. 5, process 500 may include determining, based on the historical interaction information, an impact score associated with an attribute of a first group of the users interacting in a stage of the service (block 520). The impact score may be indicative of whether the attribute is associated with corresponding interactions of the previous service interaction involving the degradation of the performance metric.

As further shown in FIG. 5, process 500 may include analyzing, based on the impact score satisfying a degradation threshold, a second group of the users to diagnose a cause of a member of the first group experiencing the degradation during one of the previous service interactions (block 530). As further shown in FIG. 5, process 500 may include identifying a difference associated with a parameter involving the first group receiving the service and the second group receiving the service (block 540). As further shown in FIG. 5, process 500 may include performing, based on the difference satisfying a difference threshold, an action associated with the parameter and the first group of the users (block 550).

Although FIG. 5 shows example blocks of process 500, in some implementations, process 500 may include additional blocks, fewer blocks, different blocks, or differently arranged blocks than those depicted in FIG. 5. Additionally, or alternatively, two or more of the blocks of process 500 may be performed in parallel.

The foregoing disclosure provides illustration and description, but is not intended to be exhaustive or to limit the implementations to the precise forms disclosed. Modifications may be made in light of the above disclosure or may be acquired from practice of the implementations.

As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code—it being understood that software and hardware can be used to implement the systems and/or methods based on the description herein.

As used herein, satisfying a threshold may, depending on the context, refer to a value being greater than the threshold, greater than or equal to the threshold, less than the threshold, less than or equal to the threshold, equal to the threshold, not equal to the threshold, or the like.

Although particular combinations of features are recited in the claims and/or disclosed in the specification, these combinations are not intended to limit the disclosure of various implementations. In fact, many of these features may be combined in ways not specifically recited in the claims and/or disclosed in the specification. Although each dependent claim listed below may directly depend on only one claim, the disclosure of various implementations includes each dependent claim in combination with every other claim in the claim set. As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiple of the same item.

No element, act, or instruction used herein should be construed as critical or essential unless explicitly described as such. Also, as used herein, the articles “a” and “an” are intended to include one or more items, and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, or a combination of related and unrelated items), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

What is claimed is:

1. A system for identifying and diagnosing a degradation of a performance metric associated with a service, the system comprising:
one or more memories; and
one or more processors, communicatively coupled to the one or more memories, configured to:
maintain historical interaction information associated with previous service interactions involving users that received the service;
determine, based on the historical interaction information, an impact score associated with an attribute of a first group of the users interacting in a stage of the service,
wherein the impact score is indicative of whether the attribute is associated with corresponding interactions of the previous service interaction involving the degradation of the performance metric;
analyze, based on the impact score satisfying a degradation threshold, a second group of the users to diagnose a cause of a member of the first group experiencing the degradation during one of the previous service interactions;
identify a difference associated with a parameter involving the first group receiving the service and the second group receiving the service; and
perform, based on the difference satisfying a difference threshold, an action associated with the parameter and the first group of the users.

2. The system of claim 1, wherein the difference associated with the parameter is indicative of a higher percentage of the second group utilizing a system for receiving the service than the first group,
wherein the action is performed to instruct another member associated with the first group to utilize the system.

3. The system of claim 1, wherein the attribute is a first attribute and the second group is associated with a second attribute,
wherein a range of values of the attribute is mutually exclusive from a range of values of the second attribute.

4. The system of claim 1, wherein the service involves multiple stages, and
wherein the performance metric is associated with a particular stage of the multiple stages.

5. The system of claim 1, wherein the one or more processors are further configured to:
prior to performing the action, receive a request for the service from a user that is associated with the attribute; and
determine, based on the attribute, that the user is associated with the first group,
wherein the action is performed to provide a notification that indicates that the user is to receive the service using a system or process associated with the parameter.

6. The system of claim 1, wherein the one or more processors, to perform the action, are configured to:
provide, to an agent device, a notification that indicates that members associated with the attribute are to receive the service in association with a system that is associated with the parameter or a process that is associated with the parameter.

7. The system of claim 1, wherein the one or more processors, to perform the action, are configured to:
request, via a user device associated with a member of the first group, feedback associated with the member receiving the service during a corresponding service interaction; and
update, based on receiving a response associated with the feedback, an impact analysis model that is configured to determine the impact score.

8. The system of claim 1, wherein a value of the difference threshold is based on a type of the parameter.

9. A method for diagnosing a degradation of a performance metric of a service, comprising:
collecting, by a device, historical interaction information associated with previous service interactions involving users that received the service;
determining, by the device and based on the historical interaction information, an impact score associated with an attribute of a group of the users satisfies a degradation threshold;
receiving, by the device, a request involving a user receiving the service,
wherein the request indicates that the user is associated with the attribute;
determining, by the device and based on the user being associated with the attribute and the impact score satisfying the degradation threshold, a parameter that reduces a probability that the user experiences the degradation of the performance metric when receiving the service; and
performing, by the device, an action associated with the parameter and the user.

10. The method of claim 9, wherein the parameter is identified based on an analysis of a second group of the users that received the service in association with the parameter during one or more of the previous service interactions,
wherein the second group of the users did not experience the degradation of the performance metric.

11. The method of claim 9, wherein the performance metric corresponds to a length of a period of time associated with a particular stage associated with one or more of the previous service interactions.

12. The method of claim 9, wherein performing the action comprises:
providing, to an agent device, a notification that indicates that the user is to receive the service in association with the parameter.

13. The method of claim 9, comprising:
requesting, via a user device associated with the user, feedback associated with the user receiving the service in association with the parameter; and
updating, based on receiving a response associated with the feedback, an impact analysis model that is configured to determine the impact score.

14. The method of claim 9, wherein the impact score is determined using a linear regression analysis of attributes of the users.

15. A non-transitory computer-readable medium storing a set of instructions, the set of instructions comprising:
one or more instructions that, when executed by one or more processors of a device, cause the device to:
receive a request associated with a user receiving a service,
wherein the request includes service information that identifies the service and user information that identifies an attribute of the user;
determine, based on previous service interactions, an impact score associated with the attribute and a performance metric associated with providing the service,
wherein the impact score is determined based on historical service information that indicates that a first group of the users were associated with the attribute when receiving the service;
select, based on the impact score satisfying a degradation threshold and the historical service information, a parameter associated with providing the service; and
cause a provider system to be configured in association with the parameter to provide the service for the user.

16. The non-transitory computer-readable medium of claim 15, wherein the impact score is indicative of whether the attribute is associated with corresponding interactions of the previous service interaction involving the degradation of the performance metric.

17. The non-transitory computer-readable medium of claim 15, wherein the attribute comprises at least one of:
an age range of members of the first group;
a health status of members of the first group; or
a location associated with members of the first group.

18. The non-transitory computer-readable medium of claim 15, wherein the service involves multiple stages, and
wherein the performance metric is associated with a particular stage of the multiple stages.

19. The non-transitory computer-readable medium of claim 15, wherein the parameter is identified based on the historical service information indicating that a second group of the users received the service in accordance with the parameter without experiencing the degradation of the performance metric.

20. The non-transitory computer-readable medium of claim 19, wherein the attribute is a first attribute and the second group is associated with a second attribute,
wherein a range of values of the attribute is mutually exclusive from a range of values of the second attribute.
